SEQUENCE LISTING (32 sequences)

SEQ ID NO: 1 - DNA, 78 bp, Artificial Sequence
  synthesized_E-cadherin aptamer
accttggctgtcgtgttgtgctctgagcctagcttgaccacttttctttattcgctctgaggtcagtggtcagagcgt

SEQ ID NO: 2 - DNA, 25 bp, Artificial Sequence
  synthesized_Mucin 5AC
gcagttgatcctttggataccctgg

SEQ ID NO: 3 - RNA, 24 nt, Artificial Sequence
  synthesized_hsa-miR-182-5p
uuuggcaaugguagaacucacacu

SEQ ID NO: 4 - DNA, 50 bp, Artificial Sequence
  synthesized_hsa-miR-182-5p RT primer
gttggctctggtgcagggtccgaggtattcgcaccagagccaacagtgtg

SEQ ID NO: 5 - DNA, 22 bp, Artificial Sequence
  synthesized_hsa-miR-182-5p F primer
gttgtttggcaatggtagaact

SEQ ID NO: 6 - RNA, 22 nt, Artificial Sequence
  synthesized_hsa-miR-127-3p
ucggauccgucugagcuuggcu

SEQ ID NO: 7 - DNA, 50 bp, Artificial Sequence
  synthesized_hsa-miR-127-3p RT primer
gttggctctggtgcagggtccgaggtattcgcaccagagccaacagccaa

SEQ ID NO: 8 - DNA, 19 bp, Artificial Sequence
  synthesized_hsa-miR-127-3p F primer
gttttcggatccgtctgag

SEQ ID NO: 9 - RNA, 22 nt, Artificial Sequence
  synthesized_hsa-miR-183-5p
uauggcacugguagaauucacu

SEQ ID NO: 10 - DNA, 50 bp, Artificial Sequence
  synthesized_hsa-miR-183-5p RT primer
gttggctctggtgcagggtccgaggtattcgcaccagagccaacagtgaa

SEQ ID NO: 11 - DNA, 19 bp, Artificial Sequence
  synthesized_hsa-miR-183-5p F primer
gggtatggcactggtagaa

SEQ ID NO: 12 - RNA, 21 nt, Artificial Sequence
  synthesized_hsa-miR-143-3p
ugagaugaagcacuguagcuc

SEQ ID NO: 13 - DNA, 50 bp, Artificial Sequence
  synthesized_hsa-miR-143-3p RT primer
gttggctctggtgcagggtccgaggtattcgcaccagagccaacgagcta

SEQ ID NO: 14 - DNA, 19 bp, Artificial Sequence
  synthesized_hsa-miR-143-3p F primer
gtggtgagatgaagcactg

SEQ ID NO: 15 - RNA, 23 nt, Artificial Sequence
  synthesized_hsa-miR-181a-5p
aacauucaacgcugucggugagu

SEQ ID NO: 16 - DNA, 50 bp, Artificial Sequence
  synthesized_hsa-miR-181a-5p RT primer
gttggctctggtgcagggtccgaggtattcgcaccagagccaacactcac

SEQ ID NO: 17 - DNA, 19 bp, Artificial Sequence
  synthesized_hsa-miR-181a-5p F primer
ggaacattcaacgctgtcg

SEQ ID NO: 18 - RNA, 22 nt, Artificial Sequence
  synthesized_hsa-let-7a-5p
ugagguaguagguuguauaguu

SEQ ID NO: 19 - DNA, 50 bp, Artificial Sequence
  synthesized_hsa-let-7a-5p RT primer
gttggctctggtgcagggtccgaggtattcgcaccagagccaacaactat

SEQ ID NO: 20 - DNA, 19 bp, Artificial Sequence
  synthesized_hsa-let-7a-5p F primer
gggtgaggtagtaggttgt

SEQ ID NO: 21 - RNA, 22 nt, Artificial Sequence
  synthesized_hsa-let-7f-5p
ugagguaguagauuguauaguu

SEQ ID NO: 22 - DNA, 50 bp, Artificial Sequence; identical to SEQ ID NO: 19
  synthesized_hsa-let-7f-5p RT primer

SEQ ID NO: 23 - DNA, 22 bp, Artificial Sequence
  synthesized_hsa-let-7f-5p F primer
gtttggtgaggtagtagattgt

SEQ ID NO: 24 - RNA, 22 nt, Artificial Sequence
  synthesized_hsa-miR-199a-3p
acaguagucugcacauugguua

SEQ ID NO: 25 - DNA, 50 bp, Artificial Sequence
  synthesized_hsa-miR-199a-3p RT primer
gttggctctggtgcagggtccgaggtattcgcaccagagccaactaacca

SEQ ID NO: 26 - DNA, 19 bp, Artificial Sequence
  synthesized_hsa-miR-199a-3p F primer
gggacagtagtctgcacat

SEQ ID NO: 27 - RNA, 22 nt, Artificial Sequence
  synthesized_hsa-miR-29a-5p
acugauuucuuuugguguucag

SEQ ID NO: 28 - DNA, 50 bp, Artificial Sequence
  synthesized_hsa-miR-29a-5p RT primer
gttggctctggtgcagggtccgaggtattcgcaccagagccaacctgaac

SEQ ID NO: 29 - DNA, 22 bp, Artificial Sequence
  synthesized_hsa-miR-29a-5p F primer
gttgggactgatttcttttggt

SEQ ID NO: 30 - DNA, 19 bp, Artificial Sequence
  synthesized_hsa-miR-10a
ggtaccctgtagatccgaa

SEQ ID NO: 31 - DNA, 50 bp, Artificial Sequence
  synthesized_hsa-miR-10a RT primer
gttggctctggtgcagggtccgaggtattcgcaccagagccaaccacaaa

SEQ ID NO: 32 - DNA, 16 bp, Artificial Sequence
  synthesized_hsa-miR-10a F primer
gtgcagggtccgaggt

(19) United States
(12) Patent Application Publication
(10) Pub. No.: US 2020/0072843 A1
(43) Pub. Date: Mar. 5, 2020

(54) METHODS OF DIAGNOSING DISEASES BY EXTRACELLULAR VESICLES AND USES THEREOF

(71) Applicant: MacKay Memorial Hospital, Taipei City (TW)

(72) Inventors: Kuen-Der YANG, Taichung City (TW); Chie-Pein CHEN, Taipei City (TW)

(73) Assignee: MacKay Memorial Hospital, Taipei City (TW)

(21) Appl. No.: 16/556,255

(22) Filed: Aug. 30, 2019

Related U.S. Application Data
(60) Provisional application No. 62/724,645, filed on Aug. 30, 2018.

(51) Int. Cl. G01N 33/574; G01N 33/68; C12Q 1/6886; C12Q 1/6883

(52) U.S. Cl. CPC G01N 33/57488; G01N 33/6896; G01N 33/6893; C12Q 2600/178; C12Q 1/6883; C12Q 2600/158; C12Q 1/6886

(57) ABSTRACT

Disclosed herein is a method of treating a cancer, a degenerative disease, an infectious disease, or aging in a subject. According to certain embodiments of the present disclosure, the method comprises, (a) obtaining a biological sample from the subject; (b) isolating a plurality of extracellular vesicles (EVs) from the biological sample; (c) determining the expression level of a target molecule of the plurality of EVs; and (d) treating the cancer, the degenerative disease, the infectious disease, or the aging based on the expression level of the target molecule determined in step (c).

### CROSS-REFERENCE TO RELATED APPLICATION

This application relates to and claims the benefit of U.S. Provisional Application No. 62/724,645, filed Aug. 30, 2018; the content of the application is incorporated herein by reference in its entirety.

### BACKGROUND OF THE INVENTION

### 1. Field of the Invention

The present disclosure in general relates to the field of disease prediction and treatment. More particularly, the present disclosure relates to methods of identifying a subject in need of treatment via determining the expression level of one or more target molecules in extracellular vesicles (EVs) of the subject, and based on the identified results, predicting the disease and administering to the subject a suitable treatment.

### 2. Description of Related Art

Extracellular vesicle (EV), a cell-derived membrane vesicle, is present in various sources of tissue fluids, including blood, urine, cerebrospinal fluid, pleural fluid, ascites, breast milk, amniotic fluid, birth canal, gastrointestinal tract, bronchoalveolar lavage, and saliva. EVs may be released from a donor cell (such as an endothelial cell, an epithelia cell, a lymphocyte, or a cancer cell), or a remote tissue (e.g., brain, heart, pancreas, bone marrow, lung, kidney, or placenta); and then up-taken by a recipient cell via endocytosis, membrane fusion, or specific ligand-receptor internalization, thereby delivering the content of EVs (e.g., the nucleic acid, lipopolysaccharide, protein and/or lipid) from the donor cell to the recipient cell. It is known that EVs mediate various physiological and pathological responses, including organ development, cell-cell communication, tumor metastasis, and the development and progression of immune-related diseases (e.g., autoimmune diseases, degenerative diseases, or inflammatory diseases).

Since the content of EVs reflects the status of the donor cell, they may serve as a candidate molecule in the application of diagnosis, prognosis, and epidemiology. However, based on the complexity and unpredictability of the physiological and pathological responses, there remains a need to identify specific EV biomarkers associated with diseases (e.g., cancers, degenerative diseases, or infectious diseases) and/or conditions (e.g., aging), so that a skilled artisan may accurately predict or diagnose the occurrence of diseases or conditions via analyzing these EV biomarkers, and promptly administering to the subject in need thereof a suitable treatment.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a method of making a diagnosis or prognosis of a cancer, a degenerative disease, an infectious disease, or aging from a biological sample of a subject. The method comprises,

(a) isolating a plurality of extracellular vesicles (EVs) from the biological sample;

(b) determining the expression level of a target molecule of the plurality of EVs; and

(c) making the diagnosis or prognosis of the cancer, the degenerative disease, the infectious disease, or the aging based on the expression level of the target molecule determined in step (b), in which the expression level of the target molecule of the plurality of EVs different from that of a reference sample obtained from a healthy subject indicates that the subject has or is at risk of developing the cancer, the degenerative disease or the infectious disease, or is in an aging state.

In the present disclosure, the expression patterns of one or more target molecules are genuinely designed and detected by bispecific and multi-specific modules with unique antibody or/and novel aptamer that exhibit binding affinity to the one or more target molecules, and are employed to amplifying signal intensity.

According to embodiments of the present disclosure, in the diagnosis or prognosis of the cancer, the target molecule in EVs is selected from the group consisting of E-cadherin, inducible costimulator-ligand (ICOS-L), dermcidin, cell growth regulator with EF-hand domain 1 (CGREF1), cochlin, amphiregulin (AREG), leucin-rich alph2-glycoprotein (LRG), guanine deaminase, S100 calcium-binding protein A8 (S100A8), mucin 5AC (Muc5AC), neutrophil-gelatinase associated lipocalin (NGAL) hsa-miR-10a-5p, hsa-miR-182-5p, hsa-miR-10b-5p, hsa-miR-22-3p, hsa-miR-181a-5p, hsa-miR-21-5p, hsa-miR-143-3p, hsa-miR-12′7-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, hsa-miR-186-5p, and a combination thereof.

In the diagnosis or prognosis of the degenerative disease, the target molecule in EVs is selected from the group consisting of epidermal growth factor (EGF), fractalkine, α-synuclein, L1 cell adhesion molecule (L1CAM), interferon-alpha (IFN-α), IFN-γ, growth-regulated protein (GRO), interleukin (IL)-10, monocyte-chemotactic protein 3 (MCP-3), exosome DNA (exoDNA), and a combination thereof.

In the diagnosis or prognosis of the infectious disease, the target molecule in EVs is a nucleic acid, lipopolysaccharide, lipid and/or protein, such as non-structural protein 1 (NS-1).

In the diagnosis or prognosis of the aging, the targeting molecule in EVs is selected from the group consisting of α-synnuclein, L1CAM, S100A8, EGF, granulocyte-colony stimulating factor (G-CSF), granulocyte-macrophage colony-stimulating factor (GM-CSF), GRO, interleukin-1 receptor antagonist (IL-1RA), IL-1α, IL-4, IL-6, IL-8, interferon gamma-induced protein 10 (IP-10), monocyte-chemotactic protein 1 (MCP-1), macrophage inflammatory protein-1 beta (MIP-1β), tumor necrosis factor-alpha (TNF-α), basic fibroblast growth factor (bFGF), fractalkine, IFN-α, IFN-γ, macrophage-derived chemokine (MDC), exoDNA, and a combination thereof.

According to embodiments of the present disclosure, each of the plurality of EVs is characterized in having at least one marker expressed therein and/or thereon, wherein the marker is selected from the group consisting of CD9, CD63, CD81, heat shock protein 60 (HSP60), HSP90 and HSP105; and having a particle size ranging between 30 to 450 nm.

Examples of the cancer suitable to be diagnosed or predicted by the present method include, but are not limited to, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, and head and neck carcinoma.

Non-limiting examples of the degenerative disease suitable to be diagnosed or predicted by the present method include, Parkinson's disease, Alzheimer's disease, dementia, stroke, chronic kidney disease, chronic lung disease, benign prostate hypertrophy, and hearing loss.

The infectious disease suitable to be diagnosed or predicted by the present method may be caused by a bacterium, a virus, or a fungus.

According to certain embodiments of the present disclosure, the biological sample is blood, urine, cerebrospinal fluid, pleural fluid, ascites, breast milk, amniotic fluid, birth canal, gastrointestinal tract, bronchoalveolar lavage, or saliva.

According to some examples of the present disclosure, for the purpose of making a diagnosis or prognosis of the cancer, the biological sample is a urine sample isolated from the subject, and the target molecule in EVs is selected from the group consisting of E-cadherin, S100A8, Muc5AC, and a combination thereof, in which the expression level of the target molecule higher than that of the reference sample indicates that the subject has or is at risk of developing the cancer.

According to some examples of the present disclosure, the target molecule in EVs is employed to make a diagnosis or prognosis of the cancer, in which the expression level of E-cadherin, ICOS-L, Muc5AC, dermcidin, CGREF1, cochlin, AREG, LRG, guanine deaminase, S100A8, NGAL, hsa-miR-10a-5p or hsa-miR-182-5p higher than that of the reference sample, and/or the expression level of hsa-miR-10b-5p, hsa-miR-22-3p, hsa-miR-181a-5p, hsa-miR-21-5p, hsa-miR-143-3p, hsa-miR-127-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, hsa-miR-186-5p lower than that of the reference sample indicates that the subject has or is at risk of developing the cancer.

According to certain examples of the present disclosure, the biological sample for making a diagnosis or prognosis of the degenerative disease is a blood sample, a saliva sample or a urine sample isolated form the subject, and the target molecule in EVs is selected from the group consisting of EGF, fractalkine, L1CAM, α-synuclein, IFN-α, IFN-γ, GRO, IL-10, MCP-3, exoDNA, and a combination thereof, in which the expression level of EGF, fractalkine, IFN-α, IFN-γ, GRO, IL-10, or MCP-3 lower than that of the reference sample, and/or the expression level of L1 CAM, α-synuclein, or exoDNA higher than that of the reference sample indicates that the subject has or is at risk of developing the degenerative disease.

According to certain examples of the present disclosure, the biological sample for making a diagnosis or prognosis of the infectious disease is a blood or urine sample isolated from the subject, in which the target molecule in EVs is a nucleic acid, lipopolysaccharide, or microbial protein, such as NS-1, and the expression level of the target molecule higher than that of the reference sample indicates that the subject has or is at risk of developing the infectious disease.

According to some embodiments of the present disclosure, the method is used to make a diagnosis or prognosis of aging in a subject (i.e., diagnosing or predicting the aging state of a subject). In one working example, the biological sample is a blood sample isolated from the subject, and the targeting molecule in EVs is selected from the group consisting of L1CAM, S100A8, α-synuclein, dipeptidyl peptidase 4 (DPP4), CD90, ephrin receptor A2 (EphA2), IL-2, IL-12, brain-derived neurotropic factor (BDNF), b2-microglobulin (B2M), connective tissue growth factor (CTGF), neurofilament light chain (NFL), EGF, G-CSF, GM-CSF, GRO, IL-1RA, IL-6, IL-8, IP-10, MCP-1, MIP-1β, TNF-α, and a combination thereof, in which the expression level of EGF lower than that of the reference sample, and/or the expression level of G-CSF, GM-CSF, GRO, IL-1RA, IL-6, IL-8, IP-10, MCP-1, TNF-α higher than that of the reference sample indicates that the subject is in an aging state. Alternatively, the biological sample may be the urine sample of the subject, and the targeting molecule in EVs is selected from the group consisting of S100A8, DPP4, CD90, EphA2, L1CAM, IL-2, IL-12, BDNF, B2M, CTGF, NFL, EGF, bFGF, G-CSF, fractalkine, IFN-α, IFN-γ, MDC, IL-1RA, IL-1α, IL-4, IL-6, IL-8, GRO, IP-10, MCP-1, L1CAM, α-synuclein, exoDNA, and a combination thereof, in which the expression level of EGF, bFGF, G-CSF, fractalkine, IFN-α, IFN-γ, MDC, IL-1RA, IL-1α, or IL-4 lower than that of the reference sample, and/or the expression level of S100A8, IL-6, IL-8, GRO, IP-10, MCP-1, L1CAM, α-synuclein, or exoDNA higher than that of the reference sample indicates that the subject is in an aging state.

Based on the diagnostic or prognostic results, a skilled artisan may administer to the subject suffering from or having a risk of developing a cancer, degenerative disease, or infectious disease, or the subject in an aging state, an effective amount of a therapeutic agent, e.g., an anti-cancer agent, an anti-degenerative agent, an anti-infectious agent (for example, an anti-bacterial, ant-viral or anti-fungal agent), or an-anti-aging agent so as to prevent or inhibit the occurrence and/or progression the disease/condition. Accordingly, another aspect of the present disclosure provides a method of treating a cancer, a degenerative disease, an infectious disease, or aging in a subject. The method comprises,

(a) obtaining a biological sample from the subject;

(b) isolating a plurality of EVs from the biological sample;

(c) determining the expression level of a target molecule of the plurality of EVs; and

(d) treating the cancer, the degenerative disease, the infectious disease, or the aging based on the expression level of the target molecule determined in step (c), in which when the expression level of the target molecule of the plurality of EVs is different from that of a reference sample obtained from a healthy subject, then administering to the subject an effective amount of a therapeutic agent.

The features of the treating method are quite similar to that of diagnostic/prognostic method as described above, and hence, detailed description thereof is omitted herein for the sake of brevity.

The subject of the present methods is preferably a mammal. According to some working examples of the present disclosure, the subject is a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is the result of western blot assay that depicts the expression of specific markers in EV or flow-through fractions according to Example 1 of the present disclosure. PEV: EVs isolated from plasma samples. UEV: EVs isolated from urine samples. MEV: EVs isolated from mesenchymal stem cells of umbilical cord. SEV: EVs isolated from saliva samples. Soln: flow-through fraction of plasma samples, urine samples, mesenchymal stem cells of umbilical cord, or saliva samples.

FIG. 2A depicts the result of immunoblot assay according to Example 2.1 of the present disclosure, in which compared with EVs isolated from mesenchymal stem cells of umbilical cord (i.e., MEV), EVs isolated from the DLD-1 cancer cells (i.e., DEV) had higher levels of E-cadherin and S100A8 expression.

FIGS. 2B and 2C are histograms respectively depicting the miRNA profiles of MEV and DEV according to Example 2.1 of the present disclosure.

FIG. 3 depicts the results of immunochromatography according to Example 2.2 of the present disclosure, in which the expression levels of E-cadherin and Mucin 5AC in the EVs of blood isolated from healthy subject (designated as “control” in the left panel of FIG. 3) were lower than those in the EVs isolated from the blood of cancer patients (designated as “cancer” in the right panel of FIG. 3). The detection was performed by a bispecific capture of EVs, in which anti-CD63 antibody (0.2 mg/ml) at 1:2,000 dilution was incubated with EVs in the sample loading region, and the aptamers of E-cadherin and Mucin 5AC were separately labeled in the upper pole of immunochromatography strip. The bispecific binding was developed by a second antibody conjugated with streptavidin-HRP (1:2,000) for chemiluminescence imaging.

FIGS. 4A and 4B are photographs of immunochromatography according to Example 4 of the present disclosure, in which the NS-1 expression captured by gold nanoparticle conjugated anti-NS1 antibody could be detected in the EVs isolated from the plasma or urine of patients with dengue fever, but not in non-EV fractions (pSoln or uSoln) (FIG. 4A); five of 6 EVs derived from urine samples of dengue suspected patients revealed positive of NS1 expression (FIG. 4B). Plasma: plasma sample obtained from patient infected by dengue virus (DEV). PEV: EVs isolated from the plasma sample of DEV-infected patient. pSoln: flow-through fraction of the plasma sample obtained from DEV-infected patient. UEV: EVs isolated from the urine sample of DEV-infected patient. uSoln: flow-through fraction of the urine sample obtained from DEV-infected patient. Urine: urine sample obtained from DEV-infected patient. The NS-1 signal was indicated by an arrow. UEV1-6: UEVs respectively isolated from urine samples of dengue-suspected patient numbers 1-6.

FIGS. 5A and 5B respectively depict the results of immunoblotting and immunochromatography according to Example 5 of the present disclosure, in which the expression levels of S100A8 (FIG. 5A), and α-synuclein (FIG. 5B) in EVs of aging subject (older than 50 years old) were higher than that in EVs of young subject (younger than 50 years old). The representative results were reproducible in 3 paired experiments.

FIG. 6 is a photograph of western blot assay that depicts the expression difference between young subject, and old subject with or without Parkinson's disease according to Example 5 of the present disclosure. Young UEV: UEV isolated from the subject younger than 50 years old. Old UEV: UEV isolated from the subject older than 50 years old. PD UEV: UEV isolated from the subject older than 50 years old and having Parkinson's disease. Young uSoln: flow-through fraction of the urine sample obtained from the subject younger than 50 years old. Old uSoln: flow-through fraction of the urine sample obtained from the subject older than 50 years old. PD uSoln: flow-through fraction of the urine sample obtained from the subject older than 50 years old and having Parkinson's disease. The representative results were reproducible in 5 paired experiments.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the expression level of specific target molecule of the present invention, optionally together with other clinical characteristics, to arrive at a diagnosis (that is, the occurrence or nonoccurrence) of a cancer, a degenerative disease, an infectious disease, or aging for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

The term “risk” herein refers to the potential that a result will lead to an undesirable outcome, e.g., occurrence, progression or recurrence of a disease or a condition, for example, a cancer, degenerative disease, infectious disease, aging.

The term “administered,” “administering” or “administration” are used interchangeably herein to refer a mode of delivery, including, without limitation, intraveneously, intraarticularly, intratumorally, intramuscularly, intraperitoneally, intraarterially, intracranially, or subcutaneously administering an agent (e.g., an anti-cancer, anti-degenerative, anti-viral, or anti-aging agent) of the present invention.

“Treatment” as used herein includes preventative (e.g., prophylactic), curative or palliative treatment of a disease or condition in a mammal, particularly human; and includes: (1) preventative (e.g., prophylactic), curative or palliative treatment of a disease or condition (e.g., a cancer, degenerative disease, infectious disease, or aging) from occurring in an individual who may be pre-disposed to the disease or condition but has not yet been diagnosed as having it; (2) inhibiting a disease or condition (e.g., by arresting its development); or (3) relieving a disease or condition (e.g., reducing symptoms associated with the disease or condition).

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in cell number, grams, milligrams or micrograms or as milligrams per kilogram of body weight (mg/Kg). Alternatively, the effective amount can be expressed in the concentration of the active component (e.g., an anti-cancer, anti-degenerative, anti-viral, or anti-aging agent of the present disclosure), such as cell concentration, molar concentration, mass concentration, volume concentration, molality, mole fraction, mass fraction and mixing ratio. Persons having ordinary skills could calculate the human equivalent dose (HED) for the medicament (such as the present anti-cancer, anti-degenerative, anti-viral, or anti-aging agent) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The terms “subject” and “patient” are interchangeably used in the present disclosure, and refer to a mammal including the human species that is suitable to be predicted, diagnosed, and/or treated by the method of the present invention. The term “subject” or “patient” is intended to refer to both the male and female gender unless one gender is specifically indicated.

The term “healthy subject” refers to a subject that does not have a disease or condition, e.g., the subject not having a cancer, a degenerative disease or an infectious disease, or the subject not in an aging state (i.e., the subject younger than 50 years old). In general, the term “healthy subject” refers to a subject who has not been diagnosed as having a disease or condition, and is not presenting with two or more (e.g., two, three, four or five) symptoms associated with the disease or condition.

As used herein, the term “in an aging state” refers to a subject who is older than 50 years old, and exhibits one or more common signs and symptoms of aging, for example, hair color change, hair loss, wrinkles, increased susceptibility to infection, greater risk of heat stroke or hypothermia, and stooped posture.

### II. Description of the Invention

(i) Method of Predicting or Diagnosing Diseases or Conditions

The present disclosure is based, at least in part, on the discovery that compared with the EVs obtained from a control subject (i.e., a subject not having a cancer, a degenerative disease or an infectious disease, or a subject not in an aging state), the expression level of some bio-markers would be up-reregulated or down-regulated in and/or on the EVs obtained from a subject having a cancer, degenerative disease or infectious disease, or from a subject in an aging state (i.e., older than 50 years old). Thus, the present disclosure provides a method of making a diagnosis or prognosis of cancers, degenerative diseases, infectious diseases, or aging via determining the expression level of one or more bio-markers; and thus, a skilled artisan or a clinical practitioner may administer to the subject in need thereof a suitable treatment in time.

The method of making a diagnosis or prognosis of a cancer, a degenerative disease, an infectious disease, or aging in a subject, comprises,

(a) obtaining a biological sample from the subject;

(b) isolating a plurality of EVs from the biological sample;

(c) determining the expression level of a target molecule of the plurality of EVs;

(d) making the diagnosis or prognosis of the cancer, the degenerative disease, the infectious disease, or the aging based on the expression level of the target molecule determined in step (c), in which the expression level of the target molecule of the plurality of EVs different (i.e., higher or lower) from that of a reference sample obtained from a healthy subject indicates that the subject has or is at risk of developing the cancer, the degenerative disease or the infectious disease, or is in an aging state.

In the step (a), a biological sample is first obtained from the subject. Depending on intended uses, the biological sample may be a blood, urine, cerebrospinal fluid, pleural fluid, ascites, breast milk, amniotic fluid, birth canal, gastrointestinal tract, bronchoalveolar lavage, saliva, or other biofluids containing EVs. The subject of the present method is a mammal, for example, a human, a mouse, a rat, a hamster, a guinea pig, a rabbit, a dog, a cat, a cow, a goat, a sheep, a monkey, and a horse. According to preferred embodiments, the subject is a human.

Then, a plurality of EVs are isolated from the biological sample as described in the step (b). The EVs may be isolated by any method known in the art, such as differential centrifugation, sucrose gradient centrifugation, microfiltration, immunochromatography, antibody-coated magnetic beads, and commercial kits (e.g., EXOQUICK™). According to embodiments of the present disclosure, each of the isolated EVs is characterized in, (1) having specific markers (i.e., CD9, CD63, CD81, HSP60, HSP90 and/or HSP105) expressed therein and/or thereon; and (2) having a particle size ranging between 30 to 450 nm (i.e., each EV may have a particle size of 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440 or 450 nm); such the isolated EVs include exosomes (having a particle size ranging between 30 to 150 nm) and microvesicles (having a particle size ranging between 150 to 450 nm).

The isolated EVs are subjected to an assay thereby determining the level of one or more target molecules expressed in and/or on the EVs (step (c)), and then, a skilled artisan or a clinical practitioner may make a diagnosis or prognosis of whether the subject suffers from or is at risk of developing the disease or condition (i.e., a cancer, a degenerative disease, an infectious disease, or aging; step (d)). In the present disclosure, the expression patterns of one or more target molecules are genuinely designed and detected by bispecific and multi-specific modules with unique antibody or/and novel aptamer that exhibit binding affinity to the one or more target molecules, and are employed to amplifying signal intensity. According to embodiments of the present disclosure, the target molecule in EVs is a protein or a nucleic acid. Examples of assay for determining the expression level of the protein include, but are not limited to, western blot assay, enzyme-linked immunosorbent assay (ELISA), flow cytometry, microbead assay, immunochromatography, immunochemiluminescence, and bio-chip. Exemplary assays for determining the expression level of the nucleic acid include, but are not limited to, spectrometric analysis, polymerase chain reaction (PCR), quantitative PCR (qPCR), deoxyribonucleic acid (DNA) sequencing, and ribonucleic acid (RNA) sequencing.

As could be appreciated, the target molecule determined in the step (c) may vary with the types of the biological sample, and the diseases/conditions predicted or diagnosed by the present method. According to some embodiments of the present disclosure, the present method is used to make a diagnosis or prognosis of a cancer; in these embodiments, the target molecule in EVs is selected from the group consisting of E-cadherin, ICOS-L, Muc5AC, dermcidin, CGREF1, cochlin, AREG, LRG, guanine deaminase, S100A8, NGAL, hsa-miR-10a-5p (Accession number: MIMAT0000253), hsa-miR-182-5p (Accession number: MIMAT0000259), hsa-miR-10b-5p (Accession number: MIMAT0000254), hsa-miR-22-3p (Accession number: MIMAT0000077), hsa-miR-181a-5p (Accession number: MIMAT0000256), hsa-miR-21-5p (Accession number: MIMAT0000076), hsa-miR-143-3p (Accession number: MIMAT0000435), hsa-miR-127-3p (Accession number: MIMAT0000446), hsa-miR-221-3p (Accession number: MIMAT0000278), hsa-miR-222-3p (Accession number: MIMAT0000279), hsa-let-7i-5p (Accession number: MIMAT0000415), hsa-miR-27b-3p (Accession number: MIMAT0000419), hsa-miR-26a-5p (Accession number: MIMAT0000082), hsa-miR-100-5p (Accession number: MIMAT0000098), hsa-miR-151a-3p (Accession number: MIMAT0000757), hsa-miR-92a-3p (Accession number: MIMAT0000092), hsa-miR-21-3p (Accession number: MIMAT0004494), hsa-miR-125b-5p (Accession number: MIMAT0000423), hsa-miR-181b-5p (Accession number: MIMAT0000257), hsa-miR-31-5p (Accession number: MIMAT0000089), hsa-miR-30a-5p (Accession number: MIMAT0000087), hsa-let-7f-5p (Accession number: MIMAT0000067), hsa-miR-199a-3p (Accession number: MIMAT0000232), hsa-miR-28-3p (Accession number: MIMAT0004502), hsa-miR-148a-3p (Accession number: MIMAT0000243), hsa-miR-409-3p (Accession number: MIMAT0001639), hsa-miR-16-5p (Accession number: MIMAT0000069), hsa-miR-99b-5p (Accession number: MIMAT0000689), hsa-miR-381-3p (Accession number: MIMAT0000736), hsa-miR-25-3p (Accession number: MIMAT0000081), hsa-miR-410-3p (Accession number: MIMAT0002171), hsa-miR-29a-3p (Accession number: MIMAT0000086), hsa-miR-199b-3p (Accession number: MIMAT0004563), hsa-miR-125a-5p (Accession number: MIMAT0000443), hsa-miR-186-5p (Accession number: MIMAT0000456), and a combination thereof. In some working examples, the biological sample is a urine sample isolated from the subject, and the target molecule is selected from the group consisting of E-cadherin, S100A8, Muc5AC, α-synuclein, L1CAM, and a combination thereof, in which the expression level of the target molecule higher than that of the reference sample indicates that the subject has the cancer, or is at risk of developing the cancer. In certain examples, the expression level of E-cadherin, ICOS-L, Muc5AC, dermcidin, CGREF1, cochlin, AREG, LRG, guanine deaminase, S100A8, NGAL, hsa-miR-10a-5p or hsa-miR-182-5p higher than that of the reference sample indicates that the subject has the cancer, or is at risk of developing the cancer. In some examples, the expression level of hsa-miR-10b-5p, hsa-miR-22-3p, hsa-miR-181a-5p, hsa-miR-21-5p, hsa-miR-143-3p, hsa-miR-12′7-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, hsa-miR-186-5p lower than that of the reference sample indicates that the subject has the cancer, or is at risk of developing the cancer.

According to certain embodiments of the present disclosure, the present method is used to make a diagnosis or prognosis of a degenerative disease; in these embodiments, the target molecule in EVs is selected from the group consisting of ICOS-L, Muc5AC, dermcidin, CGREF1, cochlin, AREG, LRG, guanine deaminase, S100A8, DPP4, CD90, EphA2, IL-2, IL-12, BDNF, B2M, CTGF, NFL, NGAL, α-synuclein, L1CAM, EGF, fractalkine, IFN-α, IFN-γ, GRO, IL-10, MCP-1, MCP-3, exoDNA, and a combination thereof. In some examples, the biological sample is a blood sample or a urine sample isolated from the subject, in which the expression level of EGF, fractalkine, IFN-α, IFN-γ, GRO, IL-10, or MCP-3 lower than that of the reference sample indicates that the subject has the degenerative disease, or is at risk of developing the degenerative disease. In certain examples, the biological sample is a blood sample, a saliva sample, or a urine sample isolated from the subject, in which the expression level of exoDNA higher than that of the reference sample indicates that the subject has the degenerative disease, or is at risk of developing the degenerative disease.

According to certain embodiments of the present disclosure, the present method is useful in making a diagnosis or prognosis of an infectious disease. In some working examples, the biological sample is a urine sample isolated from the subject, in which target molecule of EVs is a microbial nucleic acid, lipopolysaccharide and/or protein such as NS-1 protein, and the expression level of the target molecule higher than that of the reference sample indicates that the subject has the infectious disease, or is at risk of developing the infectious disease.

According to alternative embodiments of the present disclosure, the present method is useful in making a diagnosis or prognosis of an aging state of the subject. In some working examples, the biological sample is a blood sample isolated from the subject, in which the expression level of EGF in EVs lower than that of the reference sample indicates that the subject is in an aging state. In certain working examples, the biological sample is a blood sample isolated from the subject, in which the expression level of G-CSF, GM-CSF, GRO, IL-1RA, IL-6, IL-8, IP-10, MCP-1, MIP-1β, or TNF-α in EVs higher than that of the reference sample indicates that the subject is in an aging state. In some working examples, the biological sample is a urine sample isolated from the subject, in which the expression level of EGF, bFGF, G-CSF, fractalkine, IFN-α, IFN-γ, MDC, IL-1RA, IL-1α, or IL-4 in EVs lower than that of the reference sample indicates that the subject is in an aging state. In some working examples, the biological sample is a urine sample isolated from the subject, in which the expression level of S100A8, DPP4, CD90, EphA2, IL-2, IL-12, BDNF, B2M, CTGF, NFL, IL-6, IL-8, GRO, IP-10, MCP-1, L1 CAM, α-synuclein, or exoDNA in EVs higher than that of the reference sample indicates that the subject is in an aging state. In some examples, the expression level of hsa-miR-10b-5p, hsa-miR-22-3p, hsa-miR-181a-5p, hsa-miR-21-5p, hsa-miR-143-3p, hsa-miR-12′7-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, hsa-miR-186-5p in EVs lower than that of the reference sample indicates that the subject is in an aging state, or is at risk of developing aging with and without degenerative diseases.

Examples of the cancer suitable to be predicted or diagnosed by the present method include, but are not limited to, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck carcinoma.

Non-limiting examples of the degenerative disease suitable to be predicted or diagnosed by the present method include, Parkinson's disease, Alzheimer's disease, dementia, stroke, chronic kidney disease, chronic lung disease, benign prostate hypertrophy, and hearing loss.

The infectious disease suitable to be predicted or diagnosed by the present method may be caused by a bacterium, a virus, or a fungus.

(ii) Method of Treating Diseases or Conditions

Also disclosed herein is a method of treating a cancer, a degenerative disease, an infectious disease, or aging in a subject based on the prognostic or diagnostic result of the method of Section (i) of the present disclosure. Specifically, the method of treating a cancer, a degenerative disease, an infectious disease, or aging in a subject comprises,

(a) obtaining a biological sample from the subject;

(b) isolating a plurality of extracellular EVs from the biological sample;

(c) determining the expression level of a target molecule of the plurality of EVs; and

(d) treating the cancer, the degenerative disease, the infectious disease, or the aging based on the expression level of the target molecule determined in step (c), in which when the expression level of the target molecule of the plurality of EVs is different (i.e., higher or lower) from that of a reference sample obtained from a healthy subject, then administering to the subject an effective amount of a therapeutic agent.

The steps (a)-(c) of the treating method are similar to that of the aforementioned method of Section (i) of the present disclosure, and hence, detailed description thereof is omitted herein for the sake of brevity.

In the step (d), a skilled artisan or a clinical practitioner may administer to a subject in need thereof an effective amount of a therapeutic agent (e.g., an anti-cancer agent, an anti-degenerative agent, an anti-viral agent, or an anti-aging agent) in accordance with expression level as determined in the step (c). Specifically, as mentioned in Section (i) of the present disclosure, a subject having a difference expression level of one or more target molecules from that of the reference sample indicates that the subject has or is at risk of developing the cancer, degenerative disease or infectious disease, or is in an aging state; accordingly, the skilled artisan or clinical practitioner may administer to such a subject a suitable treatment thereby preventing, ameliorating and/or alleviating the occurrence of or the symptoms associated with the cancer, degenerative disease, infectious disease, or aging.

In the present disclosure, the therapeutic agent is administered to a subject having a skewed expression patterns of one or more target molecules in EVs as compared to the healthy subject, in which the therapeutic agent may be an agonist (e.g., agomer) or antagonist (antagomir, antibody or aptamer) thereby correcting the skewed expression patterns of the one or more target molecules in the EVs of the subject.

Exemplary anti-cancer drugs include, but are not limited to, curcumin, interferons, cytokines (e.g., tumor necrosis factor, interferon α, interferon γ), antibodies (e.g. Herceptin (trastuzumab), T-DM1, AVASTIN (bevacizumab), ERBITUX (cetuximab), Vectibix (panitumumab), Rituxan (rituximab), and Bexxar (tositumomab)), anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin A analogs, Vitamin D3 analogs (e.g. EB 1089, CB 1093, and KH 1060), vitamin K, isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Examples of anti-degenerative agent include, but are not limited to, curcumin, branched-chain amino acid (BCAA, including leucine, isoleucine, and valine), cholinesterase inhibitor (such as donepezil (Aricept), galantamine (Razadyne), and rivastigmine (Exelon)), memantine (Namenda), omega-3 fatty acid, ginkgo, vitamin (including vitamin A, vitamin C, vitamin D, and vitamin E), levodopa, carbidopa, dopamine agonist (such as pramipexole (Mirapex), ropinirole (Requip), rotigotine (Neupro), and apomorphine (Apokyn)), monoamine oxidase inhibitor (MAO inhibitor, such as selegiline (Eldepryl, Zelapar), rasagiline (Azilect), and safinamide (Xadago)), catechol O-methyltransferase inhibitor (COMT inhibitor, such as entacapone (Comtan) and tolcapone (Tasmar)), Anticholinergic (such as benztropine (Cogentin), and trihexyphenidyl), and amantadine.

Examples of anti-infectious agent include, but are not limited to, LL37, interferon alpha, hydrophilic and hydrophobic antibiotics, and aptamers.

Examples of anti-aging agent include, but are not limited to, curcumin, coenzyme Q10, xanthophyll (e.g., astaxanthin, fucoxanthin and zeaxanthin), L-glutathione, retinoid, α-hydroxyl acids, β-hydroxyl acid and lutein. Alternatively, the anti-aging agent may be a proteoglycan, a glycoprotein or a glycolipid, which is optionally loaded into a scaffold for better tissue localization.

The therapeutic agent may be administered to the subject by a suitable route, for example, topical, mucosal (e.g. intraconjunctival, intranasal, intratracheal), oral, intraspinal (e.g. intrathecal), intravenous, intraarterial, intramuscular, subcutaneous, intraarticular, intraventrical, intracerebroventricular, intraperitoneal, intratumoral, and intra-middle ear administration.

As would be appreciated, the present method can be applied to the subject, alone or in combination with additional therapies that have some beneficial effects on the treatment of the cancer, degenerative disease, infectious disease, or aging, for example, anti-oxidant agents or immunotherapy. Depending on the intended purpose, the present method can be applied to the subject before, during, or after the administration of the additional therapies.

(iii) Use of the Target Molecule

Another aspect of the present disclosure is directed to the use of the target molecule serving as the biomarker for the manufacture of a kit. The use of the present disclosure is characterized in that,

the biomarker is a target molecule expressed in and/or on EVs; and

the kit is useful in making a diagnosis or prognosis of whether a subject has or is at risk of developing a cancer, a degenerative disease, an infectious disease, or aging; wherein

in the prognosis or diagnosis of the cancer, the target molecule is selected from the group consisting of E-cadherin, ICOS-L, Muc5AC, dermcidin, CGREF1, cochlin, AREG, LRG, guanine deaminase, S100A8, NGAL, hsa-miR-10a-5p, hsa-miR-182-5p, hsa-miR-10b-5p, hsa-miR-22-3p, hsa-miR-181a-5p, hsa-miR-21-5p, hsa-miR-143-3p, hsa-miR-12′7-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, hsa-miR-186-5p, and a combination thereof;

in the prognosis or diagnosis of the degenerative disease, the target molecule is selected from the group consisting of DPP4, CD90, EphA2, IL-2, IL-12, BDNF, B2M, CTGF, NFL, L1CAM, α-synuclein, EGF, fractalkine, IFN-α, IFN-γ, GRO, IL-10, MCP-3, nucleic acid (such as exoDNA), and a combination thereof;

in the diagnosis or prognosis of the infectious disease, the target molecule is NS-1;

in the diagnosis or prognosis of the aging, the targeting molecule is selected from the group consisting of S100A8, DPP4, CD90, EphA2, IL-2, IL-12, BDNF, B2M, CTGF, NFL, EGF, G-CSF, GM-CSF, GRO, IL-1RA, IL-1α, IL-4, IL-6, IL-8, IP-10, MCP-1, MIP-1β, TNF-α, bFGF, fractalkine, IFN-α, IFN-γ, MDC, L1CAM, α-synuclein, nucleic acid (such as exoDNA), and a combination thereof; and

the expression level of the target molecule of the plurality of EVs different (i.e., higher or lower) from that of a reference sample obtained from a healthy subject indicates that the subject has or is at risk of developing the cancer, the degenerative disease or the infectious disease, or is in an aging state.

The methods for determining the expression level of the target molecule, and then diagnosing or predicting the disease/condition in accordance with the determined level are quite similar to the above-mentioned method of Section (I) of the present disclosure, and hence, detailed description thereof is omitted herein for the sake of brevity.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

Materials and Methods

Isolating EVs from Different Cells

Mesenchymal stem cells isolated from umbilical cord (i.e., ucMSCs), and DLD-1 cancer cells at a concentration of 1×10⁵ cells/ml were respectively cultured in medium containing 10% fetal bovine serum (FBS) for 24 hours, followed by replacing with a serum-free medium. Forty-eight hours later, the serum-free medium (supernatant) were filtered by a microfiltration with 0.45 um pore size to remove cell debris and potential bacterial contamination, and then subjected to a 0.03 um filter thereby collecting and concentrating (200-fold) EVs retained on the 0.03 um filter. The thus-collected EVs had a particle size ranging between 30 to 450 nm. In the present experiment, EVs harvested from ucMSCs were designated as MEV, and EVs harvested from DLD-1 cells were designated as DEV. According to the results of nanoparticle tracking analysis, both MEV and DEV had an average of protein concentration of 1085.1 μg/ml (about 5.0×10¹⁰ to 10×10¹⁰ vesicles/ml).

Isolating EVs from Tissue Fluids

Four groups of subjects were included in the present investigation: (1) cancer patients diagnosed of having a head and neck cancer; (2) Parkinson's patients; (3) patients infected by dengue virus and had dengue fever; and (4) subjects older than 50 years old. The blood samples or urine samples were isolated from these subjects.

EVs were isolated from the urine sample (30 ml) by a series of centrifugation (100× concentration) and filtration with 0.45 um, 0.22 um and 0.02 um filter membranes. The drop-through fractions were collected for analyzing the purification efficiency.

EVs of blood were prepared from plasma by EXOQUICK™ precipitation. In brief, 0.5 ml of plasma was subjected to 0.126 ml EXOQUICK™ precipitation at 4° C. for 30 minutes before the centrifugation of 1,500×g for 30 minutes. The pellets of plasma EVs were re-suspended into 0.5 ml, which was aliquoted into 5 aliquots, and the supernatants were also collected for analyzing the purification efficiency.

Determination of Proteomic Profiles of MEV and DEV

The protein concentrations in MEV and DEV were measured by protein assay kit, and equalized proteins from a mixture of 3 batches of samples were subjected to the ITRAQ™ method (isobaric tags for relative and absolute quantification). The ITRAQ™ method is a protein quantitation method based on the peptides labelling with a compound that produces isobaric fragments. Employing Applied Biosystems ITRAQ™ Reagents (Applied Biosystems Inc., USA) provided as a set of four, isobaric (same mass) reagents: ITRAQ™ Reagent 114, ITRAQ™ Reagent 115, ITRAQ™ Reagent 116, and ITRAQ™ Reagent 117 to label the proteins of EVs, we made four reagents to allow 4-plex samples analysis in a LC/MS-MS experiment. A total of 1034 proteins was identified by this ITRAQ™, in which 11 of 1034 proteins had higher expression level (>5 fold) in DEV as compared to MEV. The eleven proteins having higher expression in DEV were E-cadherin, ISCO ligand, Muc5AC, dermcidin, CGREF1, cochlin, AREG, LRG, guanine deaminase, S100A8, and NGAL.

Determination of miRNA Profiles of MEV and DEV

RNA samples from MEV or DEV were harvested by lysis reagent, in which 700 ul of the lysis buffer was added to the MEV or DEV pellet for homogenization at room temperature for 5 minutes, followed by adding 140 ul of chloroform with vigorously shaking separation for 15 seconds. The total RNA samples were harvested by 12,000 g at 4° C. for 15 minutes. The collected RNA samples were then subjected to TRUSEQ® Small RNA Library Prep Kit, and the thus-produced RNA fragments were ligated with 5′ and 3′ adaptor at both ends, resulting in cDNA products (about 140 bp). The miRNA profiles of MEV and DEV were analyzed by next generation sequencing (NGS). The differential displays identified 36 miRNAs in EVs from MEV and DEV, in which 2 miRNA (i.e., miR-10a and miR-182a) had higher expression and 34 miRNA had lower expression in DEV as compared to MEV. Ten miRNAs were validated by RT-PCR quantification.

Western Blot Assay

Twenty micrograms of total protein of EVs sample derived from blood, saliva or urine were subject to SDS-PAGE under denature condition. After electrophoresis, the protein gels were transferred onto a nitrocellulose membrane by semi-dried transfer device. The membrane was incubated with non-fat dry milk for blocking of non-specific binding in Tris buffer (50 mM) followed by incubation with an antibody (at 1:2,000 dilution) or an aptamer (100 nM, for example, an aptamer of SEQ ID NO: 1 or 2, which were respectively specific to E-cadherin and Mucin 5Ac). After washing with Tris buffer to remove the unbound antibody or aptamer, the expression level of specific protein was detected via streptavidin-HRP (horse-radish peroxidase) chemiluminescence reaction.

Capture of Specific EVs in Tissue Fluids by Immunochemiluminescence

1-10 ml of tissue fluids were drained into the a cylinder of magnetic cell. The EVs were captured by the high affinity beads, and the unbound solution was drop through. The EVs captured by the ligand-coated magnetic beads were signal-amplified by immunoblot or affinity-linked fluorophotonic, chemiluminescent, or magnetoelectronic transduction so as to detect the expression of specific proteins (captured or detected by antibody or aptamer) or nucleic acids (e.g. miRNAs) (captured or detected by RT-PCR or aptamer).

Capture of Specific EVs in Tissue Fluids by Immunochromatography

EVs were mixed with anti-CD63, anti-CD9 or anti-CD81 antibody (at 1:1000 dilution) for 5 minutes before set for immunochromatography for 15 minutes, in which the upper pole of the strip was built with glass fiber and spot with aptamer(s) having high affinity to specific EV biomarkers. The initial mixture with aptamer(s) for labelling EVs may alternatively be replaced by spotting of specific antibodies to EV's biomarkers. For experiment, EVs (greater than 100 or 1000 vesicles (particles)) in 10 ul were mixed with 20 ul antibody (0.2 mg/ml) or aptamer (100 nM) with or without pre-incorporation with gold nanoparticles (40 nm, 20 OD) at 1:1 ratio, and spot onto the immunochromatography strip bottom region with 60 ul Tri buffer (50 mM). The macromolecules moved to the upper pole of the strip in the chromatography, where 100 nM aptamer was spot in glass fiber area for specific retention of EVs. For those with gold incorporation, 2nd antibody mediated color development was not required, and for those without gold incorporation, 2nd antibody with chemiluminescence or fluorescence was applied thereby detecting the expression level of specific EV bio-markers.

Detection of miRNA in EVs of Tissue Fluids

EVs isolated from the blood or urine sample of the subject was precipitated by EXOQUICK™ at 4:1 ratio. The EV pellets were then washed and lysed thereby releasing miRNAs contained in the EVs. The miRNAs were subjected to a reverse transcription reaction to produce cDNAs, and quantified by specific primers via quantitative PCR reaction. For example, hsa-miR-182-5p had a sequence of SEQ ID NO: 3, which may be validated by primers of SEQ ID NOs: 4 and 5; hsa-miR-127-3p had a sequence of SEQ ID NO: 6, which may be validated by primers of SEQ ID NOs: 7 and 8; hsa-miR-183-5p had a sequence of SEQ ID NO: 9, which may be validated by primers of SEQ ID NOs: 10 and 11; hsa-miR-143-3p had a sequence of SEQ ID NO: 12, which may be validated by primers of SEQ ID NOs: 13 and 14; hsa-miR-181a-5p had a sequence of SEQ ID NO: 15, which may be validated by primers of SEQ ID NOs: 16 and 17; hsa-let-7a-5p had a sequence of SEQ ID NO: 18, which may be validated by primers of SEQ ID NOs: 19 and 20; hsa-let-7f-5p had a sequence of SEQ ID NO: 21, which may be validated by primers of SEQ ID NOs: 22 and 23; hsa-miR-199a-3p had a sequence of SEQ ID NO: 24, which may be validated by primers of SEQ ID NOs: 25 and 26; hsa-miR-29a-5p had a sequence of SEQ ID NO: 27, which may be validated by primers of SEQ ID NOs: 28 and 29; and hsa-miR-10a had a sequence of SEQ ID NO: 30, which may be validated by primers of SEQ ID NOs: 31 and 32.

Detection of exoDNA in EVs of Tissue Fluids

EVs isolated from urine was subjected to Orange G spectrophotometry so as to detect the exosome DNA (exoDNA). In brief, concentrated EVs samples were put into Tris buffer at 1:4 of Eppendorf tubes, and lysed by a heat pot for 30 minutes. 20 ul of EVs solutions were mixed with 0.015% Orange G solution to stain double stranded DNA, and then diluted to 200 ul in duplicates for spectrophotometry at excitation/emission of 476/590 nm. The concentrations of exoDNA were calculated based on a standard curve made of a series of well-known DNA concentrations, and normalized by the basis of 1 mg of total EVs protein measured by BCA protein assay.

### Example 1 Characterization the Isolated EVs

The EVs isolated by the procedure as described in Materials and Methods were subjected to nanoparticle tracking analyzer and western blot analysis thereby determining the number, and the expression of tetraspanin (including CD9, CD63 and CD81) and vesicle protein (including HSP60, HSP90 and HSP105) thereof. According to the analytic results of nanoparticle tracking analyzer, the vesicle number of urine EVs (hereinafter as “UEV”) were about 0.5×10¹¹ to 5.1×10¹¹ depending on the urine concentration, and the vesicle number of plasma EVs (hereinafter as “PEV”) were about 5.4×10¹¹ to 7.8×10¹¹, in which the vesicle number in the EV fraction (i.e., the fraction retained on the filter membrane or precipitated by EXOQUICK™) was significantly higher than that of drop-through fraction (data not shown). The size of UEV and PEV ranged between 80 and 160 nm.

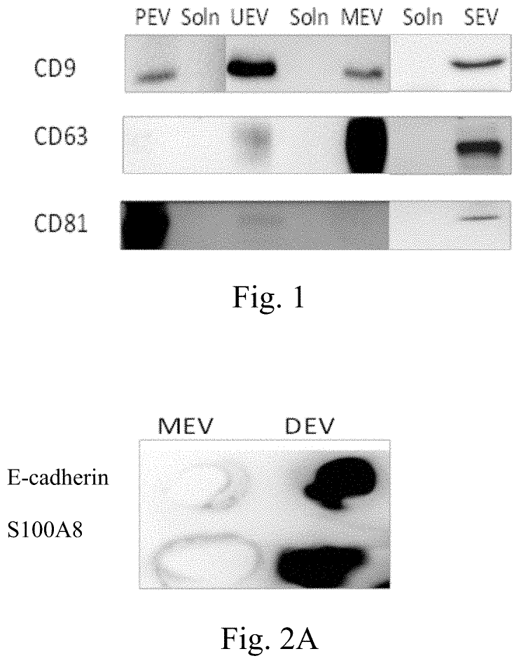

The data of western blot assay indicated that EVs isolated from plasma (i.e., PEV), urine (i.e., UEV) or saliva (i.e., SEV) had the tetraspanin (i.e., CD9, CD63 and/or CD81) (FIG. 1) and vesicle protein (i.e., HSP60, HSP90 and/or HSP105) expressed therein/thereon, in which the expression level of CD9 in UEV was higher than that in PEV, while the expression level of CD81 in PEV was higher than that in UEV (FIG. 1). No tetraspanin was detected in the drop-through fraction (FIG. 1). Based on the expression difference, CD9 and CD81 were used as the targets to respectively capture UEV and PEV in the following assays.

### Example 2 Characterization of Expression Differences Between EVs Isolated from Cancerous and Non-Cancerous Cells/Tissues

2.1 Expression Level of Target Molecules in MEV and DEV

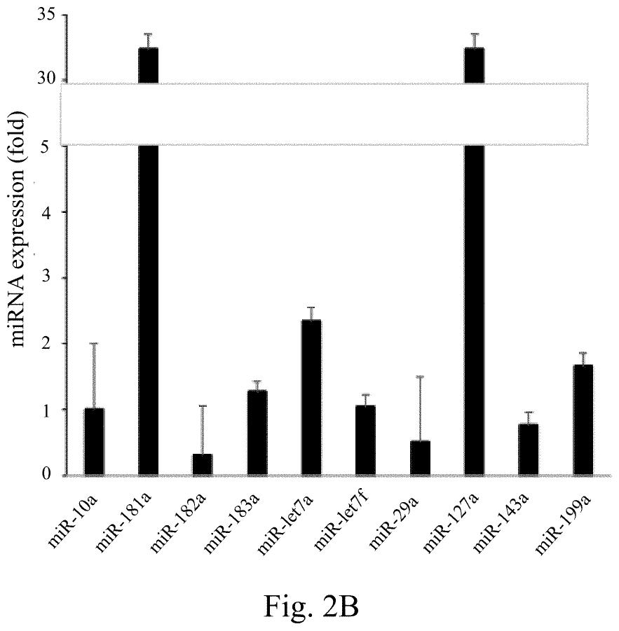

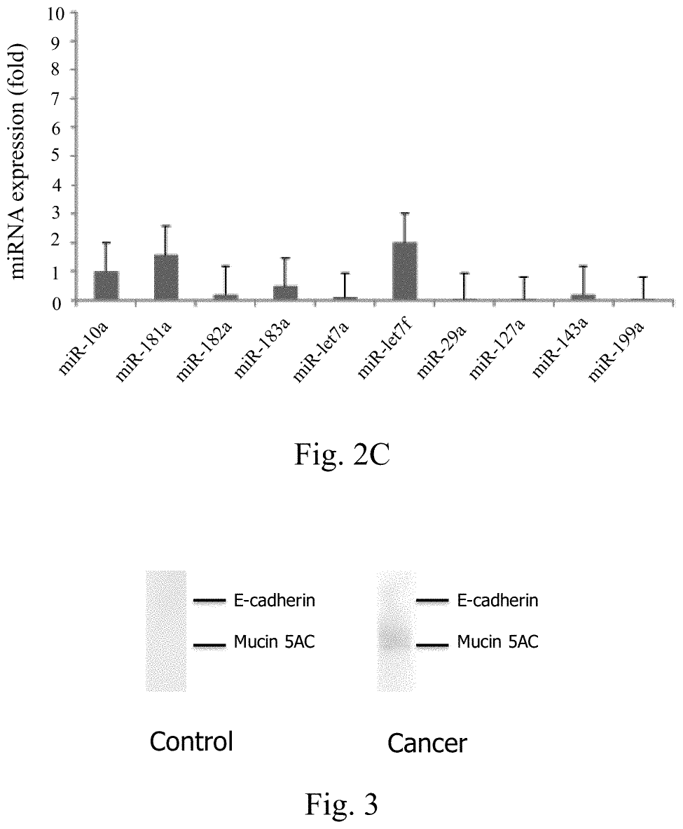

The proteomic profiles of MEV and DEV were first screen by ITRAQ™ analysis. The analytic result indicated that when the difference was defined as changes >5 fold or <⅕ fold, there were 21 proteins with different expression levels in MEV and DEV, in which 11 proteins had higher expression level in DEV as compared to MEV, including E-cadherin, ICOS-L, Muc5AC, dermcidin, CGREF1, cochlin, AREG, LRG, guanine deaminase, S100A8, and NGAL (data not shown). The data of western blot further confirmed that the expression levels of E-cadherin and S100A8 in DEV were obviously higher than that in MEV (FIG. 2A).

In addition to the proteomic profiles, the miRNA profiles of MEV and DEV were also analyzed in this example, and the results were summarized in Table 1. Compared with MEV, the levels of hsa-miR-10a-5p and hsa-miR-182-5p were up-regulated in DEV, while the levels of hsa-miR-10b-5p, hsa-miR-22-3p, hsa-miR-181a-5p, hsa-miR-21-5p, hsa-miR-143-3p, hsa-miR-12′7-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, and hsa-miR-186-5p were down-regulated in DEV (Table 1).

TABLE 1
miRNA profiles of MEV and DEV

| | DEV | MEV |
|---|---|---|
| hsamiR_ID | Fluorescent units | Fluorescent units |
| hsa-miR-10a-5p | 913.4 | 216.5 |
| hsa-miR-10b-5p | 16.1 | 126.3 |
| hsa-miR-22-3p | 1.2 | 69.4 |
| hsa-miR-181a-5p | 2.5 | 73.4 |
| hsa-miR-21-5p | 1.6 | 63.1 |
| hsa-miR-143-3p | UD | 63.5 |
| hsa-miR-127-3p | UD | 34.6 |
| hsa-miR-191-5p | 20.0 | 20.5 |
| hsa-miR-221-3p | 1.4 | 23.4 |
| hsa-miR-222-3p | 2.3 | 14.6 |
| hsa-let-7i-5p | UD | 17.2 |
| hsa-miR-27b-3p | 0.6 | 14.6 |
| hsa-miR-26a-5p | 2.2 | 12.8 |
| hsa-miR-100-5p | UD | 9.4 |
| hsa-miR-151a-3p | 2.7 | 7.3 |
| hsa-miR-92a-3p | 2.1 | 6.7 |
| hsa-miR-21-3p | UD | 13.8 |
| hsa-miR-125b-5p | UD | 5.5 |
| hsa-miR-181b-5p | UD | 9.6 |
| hsa-miR-31-5p | UD | 8.3 |
| hsa-miR-30a-5p | UD | 7.6 |
| hsa-let-7f-5p | 2.6 | 5.6 |
| hsa-miR-199a-3p | UD | 10.6 |
| hsa-miR-28-3p | 1.6 | 6.0 |
| hsa-miR-148a-3p | 1.2 | 5.8 |
| hsa-miR-409-3p | UD | 6.7 |
| hsa-miR-16-5p | UD | 8.3 |
| hsa-miR-99b-5p | UD | 4.5 |
| hsa-miR-381-3p | UD | 6.6 |
| hsa-miR-25-3p | 1.2 | 5.3 |
| hsa-miR-410-3p | UD | 4.6 |
| hsa-miR-182-5p | 8.4 | 1.0 |
| hsa-miR-29a-3p | UD | 4.5 |
| hsa-miR-199b-3p | UD | 5.3 |
| hsa-miR-125a-5p | UD | 2.0 |
| hsa-miR-186-5p | 0.5 | 3.4 |

UD: under detectable.

The quantified results of FIGS. 2B and 2C further confirmed that the expression levels of miR-181a and miR-127a in MEV were significantly higher than that in DEV.

2.2 Expression Level of Target Molecules in Subjects with or without Cancer

The expression difference as characterized in Example 2.1 was further confirmed in this example, in which the urine samples were respectively obtained from healthy subjects and cancer patients with a head and neck cancer, and EVs were then isolated from the urine samples in accordance with the procedure described in Materials and Methods. As the data of FIG. 3 depicted, compared to the control group (i.e., UEVs isolated from the healthy subject), the expression levels of E-cadherin and Mucin 5AC were obviously higher in the UEVs of cancer patients.

These results demonstrated that each of the target molecules as listed in FIGS. 2-3 and Table 1 is useful in distinguishing the cancerous and non-cancerous tissues/cells, and accordingly, may serve as a cancer marker for predicting or diagnosing the occurrence of cancers.

### Example 3 Characterization of Expression Differences Between EVs of Healthy Subjects and Parkinson's Patients

The urine samples were respectively obtained from elders (older than 50 years old) with or without Parkinson's disease, and EVs were then isolated from the urine samples as described in Materials and Methods. As the results summarized in Table 2, compared to the UEVs isolated from the healthy subjects, the expression levels of EGF, fractalkine, IFN-α, IFN-γ, GRO, IL-10, and MCP-3 were significantly lower in the UEVs of Parkinson's patients.

TABLE 2
Expression of specific target molecules in UEVs isolated from
subject with or without Parkinson's disease

| Growth factors/ | Elders, pg/ml | | Parkinson, pg/ml | |
|---|---|---|---|---|
| Cytokines | (SE) | | (SE) | |
| EGF* | 1523 | (266) | 1109 | (113) |
| FGF-2 | 6.3 | (3.3) | 5.9 | (1.6) |
| Fractalkine* | 26 | (10) | 18 | (5) |
| IFN-alpha2* | 14.5 | (4.5) | 9.0 | (1.3) |
| IFN-gamma* | 4.6 | (2.0) | 1.4 | (0.5) |
| GRO* | 41 | (19) | 16 | (5) |
| IL-10* | 271 | (89) | 77 | (35) |
| MCP-3* | 5.7 | (2.0) | 2.2 | (0.9) |

(1) * P < 0.05.
(2) Numbers of elders and patients studied were respectively 10 and 24.

The results demonstrated that EGF, fractalkine, IFN-α, IFN-γ, GRO, IL-10, and MCP-3 may be employed as a biomarker for predicting or diagnosing the occurrence of Parkinson's disease.

### Example 4 Characterization of Expression Differences Between EVs of Healthy Subjects and Patients with Dengue Fever

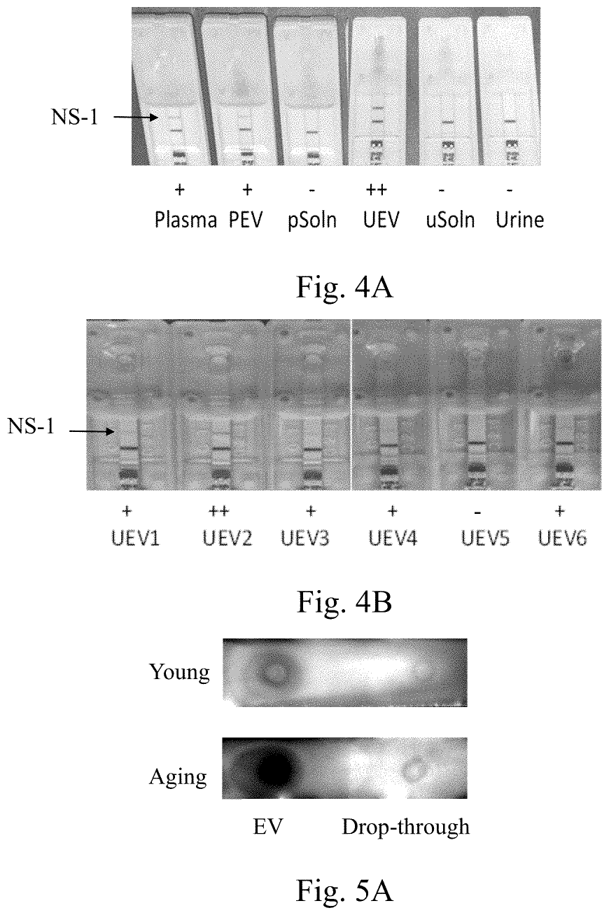

The blood and urine samples were respectively obtained from healthy subjects and patients with dengue fever. The EVs were then isolated from the blood and urine samples in accordance with the procedure described in Materials and Methods. As the data depicted in FIG. 4A, the microbial antigen NS-1 was present in PEVs and UEVs of dengue patients, while no NS-1 signal was detected in non-EV fraction (Soln). The data of FIG. 4B further indicated that 5 of 6 EVs derived from urine samples of dengue-suspected patients revealed positive of NS1 expression. The NS-1 signal detected by gold nanoparticle conjugated immunochromatography was indicated by an arrow in FIGS. 4A and 4B. The dengue NS1 protein detected in EVs of blood and urine, particularly in urine EVs, but not in non-EV fractions (pSoln or uSoln) is a novel and unique finding for prediction or diagnosis of a microbial infection (e.g., DEV infection) to our best knowledge.

### Example 5 Characterization of Expression Differences Between EVs of Young and Old Subjects

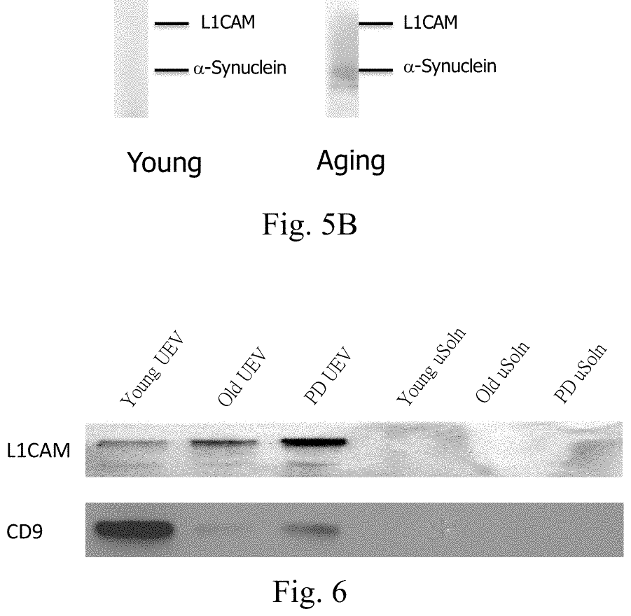

The blood and urine samples were respectively obtained from the subjects younger or older than 50 years old, and the EVs were then isolated therefrom as described in Materials and Methods. The expression difference of the isolated PEVs and UEVs was respectively summarized and depicted in Tables 3-4 and FIG. 5A-5B.

Compared to the PEVs of young subject, the expression level of EGF in elders was decreased, and the expression levels of G-CSF, GM-CSF, GRO, IL-1RA, IL-6, IL-8, IP-10, MCP-1, MIP-1β, or TNF-α were increased in the PEVs of elders (old subjects) (i.e., the subjects in an aging state) (Table 3).

TABLE 3
Expression of specific target molecules in PEVs isolated from subject
younger or older than 50 years old

| | EV (young adults) | EV (elders) |
|---|---|---|
| Conc.(pg/ml) | Mean, pg/ml (SE) | Mean, pg/ml (SE) |
| EGF | 1219 (894) | 732 (189) |
| G-CSF | 36 (21) | 155 (84) |
| GM-CSF | 8 (3) | 16 (6) |
| GRO | 3 (1) | 109 (104) |
| IL1-RA | 11 (6) | 84 (63) |
| IL-6 | 2 (1) | 123 (115) |
| IL-8 | ND | 77 (77) |
| IP-10 | ND | 2452 (640) |
| MCP-1 | 6 (4) | 716 (671) |
| MIP-1beta | 7 (2) | 65 (41) |
| TNFalpha | 15 (5) | 24 (10) |

The UEVs exhibited different expression profiles from PEVs. As the data summarized in Table 4, compared to the UEVs of young subject, the expression levels of EGF, bFGF, G-CSF, fractalkine, IFN-α, IFN-γ, MDC, IL-1α and IL-4 were decreased in elders, and the expression levels of GRO, IL-6, IL-8, IP-10 and MCP-1 were increased in the UEVs of old subjects (i.e., the subjects in an aging state).

TABLE 4
Expression of specific target molecules in UEVs isolated from subject
younger or older than 50 years old

| Growth factors/ | EV, young adults (SE) | | EV, elders (SE) | |
|---|---|---|---|---|
| Cytokines | (pg/ml) | | (pg/ml) | |
| EGF | 3319 | (392) | 1523 | (266) |
| FGF-2 | 15 | (5) | 6 | (3) |
| G-CSF | 1542 | (461) | 384 | (90) |
| Fractalkine | 86 | (42) | 26 | (11) |
| IFN-alpha2 | 40 | (19) | 15 | (5) |
| IFN-gamma | 14 | (10) | 5 | (2) |
| MDC | 162 | (44) | 48 | (19) |
| IL1-RA | 470 | (168) | 115 | (38) |
| IL-1alpha | 2.7 | (1.0) | 0.4 | (0.2) |
| IL-4 | 23 | (15) | 9 | (4) |
| GRO | 15 | (5) | 41 | (19) |
| IL-6 | 0.6 | (0.3) | 2.5 | (1.9) |
| IL-8 | 14 | (8) | 46 | (34) |
| IP-10 | 23 | (6) | 29 | (16) |
| MCP-1 | 108 | (37) | 176 | (58) |

The data of FIGS. 5A and 5B further indicated that the UEVs of subjects in an aging state (designated as “aging” in FIGS. 5A and 5B) had higher levels of S100A8 and α-synuclein, respectively, in immunoblot and immunochromatography assays as compared to the UEVs of subjects not in an aging state (designated as “young” in FIGS. 5A and 5B).

These results suggested that each of S100A8, EGF, G-CSF, GM-CSF, GRO, IL-1RA, IL-1α, IL-4, IL-6, IL-8, IP-10, MCP-1, MIP-1β, TNF-α, bFGF, fractalkine, IFN-α, IFN-γ, MDC, L1CAM, and α-synuclein may serve as a biomarker to diagnosed whether a subject is in an aging state.

The present invention further investigated the expression of L1CAM and exoDNA in UEVs of young subjects, and old subjects with or without Parkinson's disease. As the data summarized in FIG. 6 and Table 5, compared with the subject older than 50 years old, the subject younger than 50 years old had lower expression of L1CAM and exoDNA, but higher expression of CD9; the expression levels of L1CAM and exoDNA would further increase in the subject having Parkinson's disease. The reciprocal expression of L1CAM and CD9 between young and old adults could be a good predictor of aging.

TABLE 5
ExoDNA concentrations in specific subjects

| Concentration | | | |
|---|---|---|---|
| (ng/μg/mg | | | Old subjects having |
| protein) | Young subjects | Old subjects | Parkinson's disease |
| exoDNA (SE) | 118.27 | 363.09 (89.39)*^{,#} | 1042.78 (297.95)*^{,#} |
| | (50.92)* | | |

1. exoDNA is measured by Orange G spectrometry.
2. SE presents standard error (n = 10).
3. * indicates P < 0.05 and ^{#} indicates P < 0.01 between groups.

In conclusion, the present disclosure demonstrated that several target molecules exhibited different expression levels in subjects with or without specified disease or condition, including cancer, degenerative disease, infectious disease, and aging. Based on the results of the present disclosure, a skilled artisan or a medical practitioner may make a prognosis or an early diagnosis of these diseases and conditions, and thus, administering to the subject in need thereof a suitable treatment in time so as to efficiently inhibit the occurrence or progression of the disease or condition in the subject.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of treating a cancer, a degenerative disease, an infectious disease, or aging in a subject, comprising
(a) obtaining a biological sample from the subject;
(b) isolating a plurality of extracellular vesicles (EVs) from the biological sample;
(c) determining the expression level of a target molecule of the plurality of EVs, wherein
in the treatment of the cancer, the target molecule is selected from the group consisting of E-cadherin, inducible costimulator-ligand (ICOS-L), dermcidin, cell growth regulator with EF-hand domain 1 (CGREF1), cochlin, amphiregulin (AREG), leucin-rich alph2-glycoprotein (LRG), guanine deaminase, S100 calcium-binding protein A8 (S100A8), mucin 5AC (Muc5AC), neutrophil-gelatinase associated lipocalin (NGAL), hsa-miR-10a-5p, hsa-miR-182-5p, hsa-miR-10b-5p, hsa-miR-22-3p, hsa-miR-181a-5p, hsa-miR-21-5p, hsa-miR-143-3p, hsa-miR-127-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, hsa-miR-186-5p, and a combination thereof;
in the treatment of the degenerative disease, the target molecule is selected from the group consisting of epidermal growth factor (EGF), fractalkine, L1 cell adhesion molecule (L1CAM), α-synuclein, interferon-alpha (IFN-α), IFN-γ, growth-regulated protein (GRO), interleukin (IL)-10, monocyte-chemotactic protein 3 (MCP-3), exosome DNA (exoDNA), and a combination thereof;
in the treatment of the infectious disease, the target molecule is non-structural protein 1 (NS-1); and
in the treatment of the aging, the targeting molecule is selected from the group consisting of S100A8, EGF, granulocyte-colony stimulating factor (G-CSF), granulocyte-macrophage colony-stimulating factor (GM-CSF), GRO, interleukin-1 receptor antagonist (IL-1RA), IL-1α, IL-4, IL-6, IL-8, interferon gamma-induced protein 10 (IP-10), monocyte-chemotactic protein 1 (MCP-1), macrophage inflammatory protein-1 beta (MIP-1β), tumor necrosis factor-alpha (TNF-α), basic fibroblast growth factor (bFGF), fractalkine, IFN-α, IFN-γ, macrophage-derived chemokine (MDC), L1CAM, α-synuclein, exoDNA, and a combination thereof; and
(d) treating the cancer, the degenerative disease, the infectious disease, or the aging based on the expression level of the target molecule determined in step (c), in which when the expression level of the target molecule of the plurality of EVs is different from that of a reference sample obtained from a healthy subject, then administering to the subject an effective amount of a therapeutic agent.

2. The method of claim 1, wherein each of the plurality of EVs is characterized in,
having at least one marker expressed therein and/or thereon, wherein the marker is selected from the group consisting of CD9, CD63, CD81, heat shock protein 60 (HSP60), HSP90 and HSP105; and
having a particle size ranging between 30 to 450 nm.

3. The method of claim 1, wherein the cancer is gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck carcinoma.

4. The method of claim 1, wherein the degenerative disease is Parkinson's disease, Alzheimer's disease, dementia, stroke, chronic kidney disease, chronic lung disease, benign prostate hypertrophy, or hearing loss.

5. The method of claim 1, wherein the infectious disease is caused by a bacterium, a virus, or a fungus.

6. The method of claim 1, wherein the biological sample is blood, urine, cerebrospinal fluid, pleural fluid, ascites, breast milk, amniotic fluid, birth canal, gastrointestinal tract, bronchoalveolar lavage, or saliva.

7. The method of claim 6, wherein in the treatment of the cancer,
the biological sample is the urine; and
the target molecule is selected from the group consisting of E-cadherin, L1CAM, α-synuclein, S100A8, Muc5AC, and a combination thereof, wherein when the expression level of the target molecule is higher than that of the reference sample, then administering to the subject an effective amount of an anti-cancer agent.

8. The method of claim 6, wherein in the treatment of the cancer,
when the expression level of E-cadherin, ICOS-L, Muc5AC, dermcidin, CGREF1, cochlin, AREG, LRG, guanine deaminase, S100A8, NGAL, hsa-miR-10a-5p or hsa-miR-182-5p is higher than that of the reference sample; and/or
when the expression level of hsa-miR-10b-5p, hsa-miR-22-3p, hsa-miR-181a-5p, hsa-miR-21-5p, hsa-miR-143-3p, hsa-miR-12′7-3p, hsa-miR-221-3p, hsa-miR-222-3p, hsa-let-7i-5p, hsa-miR-27b-3p, hsa-miR-26a-5p, hsa-miR-100-5p, hsa-miR-151a-3p, hsa-miR-92a-3p, hsa-miR-21-3p, hsa-miR-125b-5p, hsa-miR-181b-5p, hsa-miR-31-5p, hsa-miR-30a-5p, hsa-let-7f-5p, hsa-miR-199a-3p, hsa-miR-28-3p, hsa-miR-148a-3p, hsa-miR-409-3p, hsa-miR-16-5p, hsa-miR-99b-5p, hsa-miR-381-3p, hsa-miR-25-3p, hsa-miR-410-3p, hsa-miR-29a-3p, hsa-miR-199b-3p, hsa-miR-125a-5p, or hsa-miR-186-5p is lower than that of the reference sample;
then administering to the subject an effective amount of an anti-cancer agent.

9. The method of claim 6, wherein in the treatment of the degenerative disease,
the biological sample is the blood or urine or; and
the target molecule is selected from the group consisting of EGF, fractalkine, L1CAM, α-synuclein, IFN-α, IFN-γ, GRO, IL-10, MCP-3, exoDNA, and a combination thereof, wherein
when the expression level of EGF, fractalkine, IFN-α, IFN-γ, GRO, IL-10, or MCP-3 is lower than that of the reference sample; and/or
when the expression level of L1CAM, α-synuclein, or exoDNA is higher than that of the reference sample;
then administering to the subject an effective amount of an anti-degenerative agent.

10. The method of claim 6, wherein in the treatment of the infectious disease,
the biological sample is the blood or urine; and
when the expression level of NS-1 is higher than that of the reference sample, then administering to the subject an effective amount of an anti-viral agent.

11. The method of claim 6, wherein in the treatment of the aging,
the biological sample is the blood; and
the targeting molecule is selected from the group consisting of L1CAM, α-synuclein, EGF, G-CSF, GM-CSF, GRO, IL-1RA, IL-6, IL-8, IP-10, MCP-1, MIP-1β, TNF-α, and a combination thereof, wherein
when the expression level of EGF is lower than that of the reference sample; and/or
when the expression level of L1CAM, α-synuclein, G-CSF, GM-CSF, GRO, IL-1RA, IL-6, IL-8, IP-10, MCP-1, MIP-1β, or TNF-α is higher than that of the reference sample;
then administering to the subject an effective amount of an anti-aging agent.

12. The method of claim 6, wherein in the treatment of the aging,
the biological sample is the blood or urine; and
the targeting molecule is selected from the group consisting of S100A8, EGF, bFGF, G-CSF, fractalkine, IFN-α, IFN-γ, MDC, IL-1RA, IL-1α, IL-4, IL-6, IL-8, GRO, IP-10, MCP-1, L1CAM, α-synuclein, exoDNA, and a combination thereof, wherein
when the expression level of EGF, bFGF, G-CSF, fractalkine, IFN-α, IFN-γ, MDC, IL-1RA, IL-1α, or IL-4 is lower than that of the reference sample; and/or
when the expression level of S100A8, IL-6, IL-8, GRO, IP-10, MCP-1, L1CAM, α-synuclein, or exoDNA is higher than that of the reference sample;
then administering to the subject an effective amount of an anti-aging agent.

13. The method of claim 1, wherein the subject is a human.
